(19) United States
(12) Patent Application Publication
(10) Pub. No.: US 2022/0047747 A1
(43) Pub. Date: Feb. 17, 2022

(54) LIQUID OR GEL DELIVERY DEVICES

(71) Applicant: SURFACESKINS LIMITED, Leeds West Yorkshire (GB)

(72) Inventors: Stephen RUSSELL, Harrogate North (GB); Simon Joan SCOTT-HARDEN, Kenton (GB); Christopher FOWLER, York North (GB); Steven NEILL, Farsley (GB)

(73) Assignee: SURFACESKINS LIMITED, Leeds West Yorkshire (GB)

(21) Appl. No.: 17/222,481

(22) Filed: Apr. 5, 2021

Related U.S. Application Data
(63) Continuation of application No. 15/740,282, filed on Dec. 27, 2017, now abandoned.

(30) Foreign Application Priority Data
Jun. 29, 2015 (GB) 1511397.0

(51) Int. Cl. A61L 2/18; A61L 9/04; A61L 9/12; A61L 2/22

(52) U.S. Cl. CPC A61L 2/18; A61L 9/042; A61L 2209/13; A61L 2/22; A61L 2209/135; A61L 9/127

(57) ABSTRACT

A delivery device is for intermittently delivering materials such as liquids or gels over a sustained period of time to a surface in a controlled way. The surface mountable delivery device is capable of delivering, an antimicrobial agent to protect surfaces from microbial contamination and/or to disinfect surfaces, e.g. door handles, push plates, hand rails, etc. as an aid in preventing and/or hindering the spread of infectious agents. The device uses a vertically lapped nonwoven material as the storage medium for the liquid or gel like materials within the device. A non-wicking liquid mediating layer to controls transfer of liquid from a reservoir to a contact surface of the device to minimize liquid losses. An elastomeric contact liquid delivery layer is optimized for printability. Use of physical features at the device contact surface prevents pore occlusion on user contact and aids flow of liquid to the contact user.

This application is a Continuation of U.S. patent application Ser. No. 15/740,282, filed Dec. 27, 2017, which is a National Stage Application of PCT/EP2016/065227, filed Jun. 29, 2016, which claims benefit of Serial No. 1511397.0, filed Jun. 29, 2015 in the United Kingdom and which applications are incorporated herein by reference. To the extent appropriate, a claim of priority is made to each of the above disclosed applications.

The present invention relates to devices for storing and discharging materials such as liquids or gels through a contact delivery surface of the devices in a controlled way on contact with a human or other objects. In particular, the invention relates to devices that may be mountable on an object frequently contacted by humans at the point of frequent human contact with that object and which device is capable of delivering, for example, an antimicrobial agent to the contact delivery surface of the device acting as a new contact surface for the object.

### FIELD OF INVENTION

### Background Art

It is widely recognized that there is a major problem with the spread of infectious agents from one place to the next, as people/animals and organisms touch one surface and then touch another surface. Such infectious agents include microbes (microorganisms), e.g. bacteria, fungi (including spores), viruses or prions. Contact—hence the word “contagious disease”—is the most common way that pathogens can spread from one individual to another. This transmission of microbes on the hands/skin/body of people/organisms way occur when they are in the first instance transferred onto any type of human contact surface e.g. door handle or door contact surface. Then when such contact surfaces are touched by the hands/skin/body of other persons/organisms the microbes on these contact surfaces may be picked up or transmitted to this new individual. Clearly, in a high contact and highly populated environment, such as a hospital, microbes may be rapidly transmitted to staff and patients, with potentially serious consequences for patients. This is a particular problem in hospitals and medical centres, where the transmission of nosocomial infections or Healthcare Associated Infections (HCAIs), e.g. methicillin-resistant Staphylococcus aureus (MRSA), Clostridium difficile (C. diff), norovirus by surface contact is a major cause of illness. In animal husbandry a similar problem arises in the prevention of the transmission of infectious agents when aniamals are exposed to a common contact surface during their management and handling.

Infection control typically involves the prevention and control of healthcare-associated infections in primary and community care. Hospitals take steps to prevent the transmission of HCAIs to avoid infecting patients. Measures to prevent the spread of microorganisms from one person to another involve isolation or infection control. The type of infection control or isolation required for any patient depends, inter alia, on the microorganism, where the microorganisms are found on/in an individual and the patient. The most important type of isolation required for MRSA is what is called “contact isolation”. This type of isolation requires everyone in contact with the patient to be very careful about hand washing after touching either the patient or anything in contact with the patient.

About 10% of infections in the UK's public hospitals have been estimated to be airborne. This means that approximately 90% of infections are therefore transmitted in other ways, such as through contact with surfaces and other individuals. A significant number of contact surfaces in hospitals and public buildings are vertical surfaces such as door touch plates.

Biosecurity is a term that covers the actions and measures needed to be taken to safeguard individuals from diseases caused by viral, bacterial and fungal infections. Biosecurity is essential against the fight of these contagious diseases. Thus, it is highly desirable to provide a means of reducing the spread of microorganisms, e.g. in, hospitals, (where surfaces are regularly touched by many people), doctors waiting rooms and doors, public houses including door handles and furniture, veterinary buildings and doors etc., as well as office equipment/computer keyboards/mice etc.

In addition, there are many devices and arrangements in the art for the storage and delivery of liquid and/or gel like materials to various objects and surfaces for everyday uses such as wiping, cleaning and disinfecting of surfaces. These devices may take the form of wipes or pads, which have stored within them liquid and/or gel materials for the specific need.

A number of attempts have been made to address the issues around contact surface transmission of microbes as described for example in, U.S. Pat. Nos. 6,863,960, 4,832,942, US published application US2002041824A1, U.S. Pat. No. 6,821,325 and US published application US2007049894A1.

More recent solutions are provided in published International applications WO2007135424A1 and WO2013167746A1. These references describe devices having a discrete porous contact surfaces through which material may be discharged when the contact surfaces are contacted or compressed by for example a human hand.

There are continuing challenges with such devices. One challenge is that liquid or gel may be discharged through the majority of the contact delivery surface even where no actual contact has occurred. A further challenge is that there are difficulties in ensuing control or localisation of the material discharge to the contact delivery surface. A further challenge is that the nature of contact with the contact delivery surface of the device may result in reduced discharge through the contact delivery surface at the required point of contact. There are further challenges associated with maintaining even distribution of device contents within the device during storage and/or use. There are continuing challenges in minimizing significant liquid evaporation that may occur during use as there is difficulty in controlling or preventing evaporation from the porous contact delivery surfaces. This is particularly problematic when the liquid or gel consists of a volatile material such as an alcohol, e.g. ethanol. A further challenge is printability of the contact delivery surface of the device. A yet further challenge is maintaining the physical structure of the delivery device during use.

### Disclosure of the Invention

One or more of the above indicated challenges may be addressed by one or more of the various aspects and features of the present invention. The devices of the present invention may comprise up to seven distinct components arranged appropriately to provide substantially planar devices or tubiform devices or other forms. The first is a backing layer or tray unit component, the second is an active medium for delivery to a contact delivery surface of a contact liquid delivery layer of the device, the third is an active medium storage or reservoir material, which has the primary function of storing the active medium prior to discharge from the device to a contact delivery surface of a contact liquid delivery layer of the device, the fourth is an intermediate mediating and/or wicking layer located between the reservoir material and the contact liquid delivery layer comprising the contact delivery surface, the fifth is a contact liquid delivery layer comprising the contact delivery surface, which layer may, especially in planar embodiments, be bonded to a tray unit in order to encapsulate the reservoir material, the intermediate material and the active medium, the sixth is a holster, preferably for use with planar embodiments, which is more rigid than the tray unit component when used and is designed to hold the tray unit in place at the desired location for use and to support the tray unit in order to prevent it from bowing when pressure is applied by users to the surface of the device during use and the seventh is a structural support, which is designed to assist with rearranging and returning the device structure to its pre-contact state after it has been contacted, compressed and then the contact pressure is removed.

In accordance with a first aspect the present invention there is provided a device for storing and discharging liquid or gel like materials, which device comprises vertically lapped nonwoven material as a storage medium for the liquid or gel like materials within the device.

Vertically lapped nonwovens consist of a web of nonwoven material that has been folded in on itself in a corrugated fashion to produce a concertina-like, three dimensional structure that has been thermally bonded. They may also be referred to as perpendicular laid nonwovens. Examples of such materials suitable for use in the present invention include V-Lap materials as manufactured using the V-Lap Vertical Lapping System manufactured by V-Lap PTY Ltd, Australia and as described for example in WO2006/092029, the whole contents of which are hereby incorporated by reference and STRUTO materials as manufactured using the Struto system (Struto International Inc.) as described in Chapter 2.12 in Russell S. J.: Handbook of Nonwovens, Woodhead Publishing Limited, Cambridge, England, 2007, the whole contents of which are hereby incorporated by reference.

The material that is used in the manufacture of the vertically lapped nonwoven may be any material that may be formed into a web like structure, which may then be formed into a vertically lapped nonwoven. Preferably the material is in the form of a fibre, which may be laid as a web using various web forming techniques known in the art. In a preferred embodiment the material base of the fibre used for forming the web comprises at least one synthetic polymer, preferably at least one synthetic thermoplastic polymer. The web may comprise a single type of fibre or fibres of a single material composition. In a preferred embodiment the web comprises a mixture of fibre types and/or fibre material compositions.

The fibre component may comprise synthetic thermoplastic polymer staple fibres such as for example polypropylene (PP), polyethylene (PE) and polyamide (PA), PLA, PBT, PET, coPET, copolyester elastomers, HDPE, LDPE, PPS, PEI, PETG, PCT, elastomeric fibres or mixtures thereof. The fibre component may further comprise bicomponent fibres and/or conjugate fibres such as for example polyethylene terephthalate (PET)/copolyester or PET/PE or PP/PE or Elk® (ELASTY™) binder fibre or conjugate/spiral PET. The bicomponent fibres are typically and preferably the binder component of the fibre composition. The conjugate fibres are preferably spiral fibres and preferably are spiral crimped fibres. These fibres will thus have a spring-like crimp. These conjugated fibres assist in providing the desired loft and resilience in the vertically lapped nonwoven material. The staple fibres may be used with or without hydrophilic treatment; it is preferred that they are hydrophilic treated.

The preferred ranges for the amount of staple thermoplastic fibre in the final web composition are from 5 to 65%, preferably 10 to 60%. The preferred ranges for the amount of bicomponent fibre in the final web composition are from 5 to 65%, preferably 10 to 60%. The preferred ranges for the amount of conjugate fibres in the final web composition are 0 to 60%, preferably 0 to 50%. Save that the amounts selected for each fibre component are such that the composition sums to 100% fibre.

It is preferred that the vertically lapped nonwoven has a density in the non-compressed state within the range of 20 to 90 kg·m⁻³, preferably within the range of 30 to 80 kg·m⁻³, more preferably within the range of 40 to 70 kg·m⁻³, more preferably within the range of 45 to 65 kg·m⁻³ and most preferably within the range of 50 to 60 kg·m⁻³.

The vertically lapped nonwoven material may be arranged in the device as a single layer or multiple layers. It is preferred that the vertically lapped nonwoven has a thickness within the range of 4 to 25 mm, more preferably 5 to 18 mm, more preferably 6 to 13 mm and most preferably 8 to 11 mm. The preferred thickness is around 9.5 mm±0.5 mm. These thicknesses are preferred for planar embodiments of the device, where it is also preferred that the vertically lapped nonwoven material is a single layer of that material. In none planar embodiments such as tubiform devices the vertically lapped nonwoven may be and is preferably thicker or multilayered. When present in multiple layers each layer of the vertically lapped nonwoven material may of the same or different thickness. In one tubiform embodiment the vertically lapped nonwoven material is a single layered material that is wound perpendicular to and around a central core backing layer and upon itself to form a multiple layered vertically lapped nonwoven material; in cross-section this would present as a spiral arrangement of vertically lapped nonwoven material.

It is preferred that the vertically lapped nonwoven material has a weight, at a target thickness of 9.5 mm, within the range of 200 to 900 g·m⁻², preferably within the range of 300 to 800 g·m⁻², more preferably within the range of 400 to 700 g·m⁻², more preferably within the range of 450 to 600 g·m⁻² and most preferably within the range of 475 to 575 g·m⁻². A weight of about 520 g·m⁻² is preferred. The related densities for these ranges and preferences may easily be determined at this target thickness. Weight values consistent with these ranges will vary proportionally with nominal thickness for a given density of material.

A preferred embodiment of the present invention comprises a vertically lapped nonwoven of weight at 9.5 mm of about 520 g·m⁻², preferably with a density within the range of 52 to 58 kg·m⁻³. In one embodiment the vertically lapped nonwoven preferably comprises a fibre mixture comprising 20% by weight conjugate PET, 40% by weight hydrophilic PET and 40% by weight of PET/coPET. In one preferred embodiment the conjugate PET fibres have a linear mass density of about 16.5 dtex, the hydrophilic PET fibres have a linear mass density of about 4.4 dtex and the PET/coPET fibres have a linear mass density of about 2.2 dtex. In a further preferred embodiment the vertically lapped nonwoven comprises 20% by weight of spiral PET fibres (10 or 16.5 dtex), 40% by weight of hydrophilic PET fibres (4.4 dtex) and 40% by weight of PET/coPET (2.2 dtex).

The surface of the vertically lapped nonwoven storage material facing away from the contact liquid delivery layer may have a closed surface, which as a consequence exhibits no open surface pore structure or a pore structure in which the ratio of open area to total area is no greater than 0.3. This may be achieved for example by providing one surface of the vertically lapped nonwoven material with a skinned surface that may be produced by thermal treatment of the material. The composition of the vertically lapped nonwoven material being such that under the action of heat its surface melts or flows and coalesces and then solidifies when the heat source is removed. This skinned surface of the vertically lapped nonwoven material is in contact with the tray unit surface when used, which forms the back surface of the device. The use of a skinned surface in this manner will prevent liquid or gel material from escaping from the vertically lapped nonwoven material through this back surface and is also valuable in improving the mechanical stability of the vertically lapped nonwoven material storage layer.

The device of the first aspect may in one embodiment be used for discharging liquid and or gel like materials to the surfaces of objects to be cleaned, wiped and/or disinfectant and the active material within vertically lapped nonwoven storage material may be any material suitable for the proposed use of the device.

In a preferred embodiment the device of the first aspect is device that may be mountable on an object at the location on that object, which is frequently contacted by humans and which device is capable of delivering, for example, an antimicrobial agent to a contact delivery surface of the device acting as a new contact surface for the object at that location. Thus the delivery device may comprise a vertically lapped nonwoven material as the storage material or reservoir for the liquid or gel like materials in communication with an elastic contact delivery layer with a contact delivery surface, the elastic contact delivery layer may have and preferably does have a plurality of closed slits and/or pores.

In accordance with a second aspect of the present invention there is provided a device for storing and discharging liquid or gel like materials, which device comprises a reservoir material within the device as the storage medium for the liquid or gel like materials, the reservoir material being in communication with a liquid mediating layer located between the reservoir material and liquid discharging surface of the device.

Prior art devices such as those described in WO2013167746A1, in contrast to this second aspect of the present invention, have utilized a wicking layer located between the reservoir material and the liquid contact delivery layer. These wicking layers are required to act as a liquid distribution and wicking layer by means of capillary forces, to be highly absorbent. They have a higher capillarity than the reservoir material that they are in contact with. This means that they essentially act as on intermediate store of liquid or gel like material that is absorbed from the reservoir throughout its contact with the wicking layer. The liquid or gel like material therefore being held proximate to the contact liquid delivery layer within this wicking layer

In contrast the liquid mediating layer of this second aspect of the present invention has a capillarity that is less than that of the reservoir material or vertically lapped nonwoven material. In addition, it is not an absorbent material and unlike the wicking layer is unable to absorb and store liquid or gel like material from the reservoir material or vertically lapped nonwoven material. Thus in this aspect of the present invention the liquid mediating layer when the device is not under compression in use acts as a barrier between the liquid containing reservoir or vertically lapped nonwoven material and the liquid contact delivery layer.

The use of a liquid mediating layer, which does not have wicking properties, in place of the prior used wicking layers has certain advantages. Firstly, the liquid remains in the reservoir or vertically lapped nonwoven material when there is no compression and does not significantly contact the liquid contact delivery layer; this means that there is less liquid loss during use and that the contact liquid delivery layer is not adversely affected by continuous contact with the liquid, especially those containing alcohol. Because the liquid mediating layer is devoid of liquid without compression of the device there is no or reduced liquid loss from the device. In addition, the lack of absorption, which in wicking layers resulted in the increased loss of volatile components such as alcohol, means that the composition of the liquid phase is more stable. With preferential loss of volatile components in wicking layer based systems the liquid composition could vary over time, which is undesirable.

The liquid mediating layer may be made of any highly porous and hydrophilic material that is non-absorbent with respect to reservoir or vertically lapped nonwoven materials. Alternatively, the liquid mediating layer may be made of a hydrophobic material that is non-absorbent whose surface has been modified to provide hydrophilicity. Any material that may be placed adjacent to a reservoir or vertically lapped nonwoven material, without absorbing liquid contained in those materials is suitable. The liquid mediating layer should be non-wicking in nature. The surface of the liquid mediating layer being hydrophilic allows the liquid or gel-like material stored in the reservoir or vertically lapped nonwoven materials to pass through it on compression of the device and to emerge from the liquid contact delivery layer.

Suitable mediating layer materials may be nonwoven materials such as spunbond (S) or meltblown (M) or a combination of these types of materials in SMS and SMMS nonwovens that are formed of 3 or 4 layers of spunbond and meltblown plies. Other combinations of spunbond and meltblown nonwovens are also suitable including SMMS, SM as well as others known in the art. Other suitable mediating layer materials may be needlepunched, hydroentangled, or a woven or knitted structure.

Preferred mediating layer materials are spunbond nonwoven materials. Preferably these mediating layer structures comprise thermoplastic materials such as thermoplastic polymers and polyolefins e.g. polyethylene, polypropylene or polyethylene terephthalate or polyamide and mixtures of one or more of these materials. Preferably the mediating layer structures comprise polypropylene. Preferably these materials are hydrophilic and this includes materials that have been treated to render them hydrophilic. These materials may be rendered hydrophilic through surface coating, plasma treatment, UV grafting and/or through used of hydrophilic masterbatch additives. The surface energy of the material is modified and selected so as to allow adequate wetting of its surfaces by the liquid or gel compositions used in the device; this modification will depend on the viscosity and surface tension of the liquid or gel material. Preferably these materials are of high porosity and low weight per unit area. Preferably their weight per unit area is less than 60 g·m⁻², preferably less than 50 g·m⁻², more preferably less than 40 g·m⁻² and most preferably between 10 and 30 g·m⁻². Preferably these weight values are at mediating layer thickness within the range of 0.125 to 0.2 mm and most preferably within the range of 0.15 to 0.175 mm. The layer may be meltblown or a combination of spunbond and meltblown. One preferred embodiment comprises a mediating layer of 20 g·m⁻² hydrophilic spunbond polypropylene.

Preferred mediating layer materials have a porosity (as measured by the method described at page 413 of in Russell S. J.: Handbook of Nonwovens, Woodhead Publishing Limited, Cambridge, England, 2007) of at least 80%, more preferably at least 85% and most preferably at least 88%.

It is important that the absorbency preferably moisture absorbency of the mediating layer is as low as possible so that when in contact with reservoir or VLAP layer no liquid is absorbed by the mediating layer from the reservoir or VLAP layer. Preferably the moisture absorption of this mediating layer from the reservoir or VLAP layer is less than 0.5% by weight, more preferably less than 0.3% by weight, more preferably less than 0.2% by weight and most preferably less than 0.1% by weight. Thus the mediating layer has little or no capacity to absorb moisture from the reservoir or VLAP layer under capillary forces. Despite this function and property, the mediating layer will have a relatively high absorptive capacity for water. With hydrophilic spunbond materials as mediating layers it is preferred that they are of a form and composition such that they have an absorptive capacity of at least 100%, preferably at least 150%, more preferably at least 200% and most preferably at least 250% and around 275% as determined by the Edana method WSP 010 1 R3(12). The properties of the mediating layer are such that on release of user pressure during use of the device any liquid that has been forced from the reservoir into the mediating layer on compression of the device is not retained in the mediating layer material but is reabsorbed into the reservoir material. As it is required for the liquid or gel to be able to freely pass through the mediating layer on compression of the device as indicated above the material of this layer is such that it may be easily wetted by the liquid or gel material and thus not impeding the flow of the liquid or gel from the reservoir and through its bulk to the contact delivery surface. It is preferred that the thickness of the mediating layer is less than 0.5 mm, more preferably less than 0.4 mm, more preferably less than 0.3 mm and most preferably is 0.25 mm or less and preferably between 0.05 to 0.25 mm. It is preferred that the mediating layer has an air permeability within the range of 200 to 400, preferably 225 to 350 and most preferably 250-300 cm³/cm² s⁻¹.

This second aspect of the present invention may be combined with the first aspect of the present invention. The reservoir material of the second aspect may be replaced with or supplemented with a vertically lapped nonwoven material.

One of the challenges associated with prior art devices is that liquid must be effectively discharged from the devices contact surface at the point of contact on that surface by a human or object. What has been found is that the act of contact tends to occlude the slits and/or pores in a contact liquid delivery layer directly under the point of contact with the result that liquid material is only significantly discharged around that point of contact with the resultant reduction in transfer of liquid material from the device to the person or object that has come into contact with the device contact surface.

Thus in accordance with a third aspect of the present invention there is provided a device comprising means for storing liquid or gel like materials and a contact liquid delivery layer with a contact surface through which the liquid or gel like materials may be discharged on contact of that surface with a human or object, characterized in that the contact surface of the contact liquid delivery layer comprises one or more physical features that prevent full contact by the human or object with the contact surface during their contact with the device.

It has surprisingly been found that if one or more physical features are located in an appropriate way on the contact surface of the device they are able to moderate the extent of physical contact by a human or object with that surface and through that moderation prevent occlusion of the slits and/or pores at the point of contact thereby increasing the discharge of liquid or gel like material at the point of contact and increasing the transfer of such material to the human or object in contact with the device. These physical features act in effect as a liquid distribution aid or layer upon the contact surface.

The contact moderating physical features may be any material that is discretely deposited upon and attached to the contact surface and presents an initial point of contact by a human or object with the device. The surface area density of these features is such that during contact with the device the human or object may apply enough force to compress the device and discharge liquid therefrom without occluding slits and/or pores at the general contact location. These features may for example be small islands or ridges of material that have been deposited upon and over on the contact surface area in sufficient density to provide this effect. These features may for example be a web or fabric of material deposited upon and bonded to the contact surface. In one embodiment these features may be a layer of low density material bonded to the contact surface, which apparently occludes the surface but does no occlude the slits and/or pores and allows liquid discharge. In one embodiment these features may be an integral part of the contact liquid delivery layer, which is manufactured to provide such features at its contact surface; in one embodiment these may take the form of a rippled or stippled effect on the material surface or other forms of embossed surface.

In a preferred embodiment the contact moderating physical feature is a filamentous material that is preferably heat sealed to the contact surface. Preferably it is heat sealed at its edges to the contact surface whilst it is stretched, under tension and in intimate contact with the contact surface; in this arrangement the filamentous layer material is not secured in its totality to the contact surface.

This filamentous layer may be a nonwoven structure such as spunbond, meltblown or a combination thereof, such as SMS or SMMS, needlepunched, hydroentangled, or a woven structure or a knitted structure. It may be a hydrophobic polymer such as one or more of PE, PP, PET, PA PLA, PBT, PET, coPET, copolyester elastomers, HDPE, LDPE, PPS, PEI, PETG, PCT, elastomeric fibres or mixtures thereof, preferably thermoplastic or a hydrophobic polymer that has been treated to make it hydrophilic via surface coating, plasma treatment, UV grafting, hydrophilic masterbatch additives. The contact moderating physical feature may be provided by a weft knit fabric, such as a weft knit PET fabric (Airtex or Aertex) ideally of low density and preferably less than 150 g/m², more preferably less than 125 g/m² and most preferably less than 100 g/m². This layer may also be printable.

In one embodiment a micro-perforated polymer film, preferably a thermoplastic polymer film, may attached to e.g. by heat sealing to the surface of a spunbond filamentous layer; this may be advantageous in protecting the filamentous spundbond layer from abrasion during use. This film layer may also be printable.

The preferred weight of the filamentous layer is from 5 to 100 g·m⁻², preferably 5 to 60 g·m², more preferably 5 to 40 g·m⁻² and most preferably 10 to 30 g·m⁻².

This filamentous layer may be dope dyed (to provide colour) or contain antimicrobial agents (normally provided as a component in the masterbatch prior to manufacture), or may be coated or otherwise treated to provide additional functionality in terms of antimicrobial behavior, surface energy and/or printability.

The third aspect of the present invention may be used in combination with either or both of the first and second aspects of the present invention.

One of the challenges associated with prior art devices is that during use and upon compression they are deformed and on occasion when compressive forces are removed they are unable to quickly and effectively revert to the pre-compressed state. It has been found that this problem can be alleviated by utilising a structural support within the device, which is designed to assist with reverting and returning the device structure to its pre-contact and compressed state after it has been contacted, compressed and then the contact pressure is removed.

Thus in a fourth aspect of the present invention there is provided a device for storing and discharging liquid or gel like materials, which device comprises a liquid or gel storage medium such as a reservoir and/or VLAP material and at least one structural support layer.

The structural support layer may be planar for use in planar devices or may be tubiform for use in tubiform devices. It is designed to offer no significant resistance to the discharge of liquid or gel from the liquid or gel storage medium to the exterior of the device upon compression of the device. Thus it comprises a plurality of holes of relatively large dimensions. These holes are of the order of millimeters wide and preferably have a minimum diameter when circular of 1 mm or greater, preferably 2 mm or greater, more preferably 3 mm or greater. When the holes are slots are other shapes it is preferred that their total area is equal to or greater than that of a circular hole. The size of these holes are ideally as large as possible to be able to offer the minimum restriction of fluid flow from the reservoir and/or VLAP in the bore of the structural support layer or beneath a planar layer to the exterior of the device whilst ensuring that the structural support layer is able to provide its resiliently deformable function. The material selected for the structural support layer is resiliently deformable, meaning that it is able to flex and distort under compression but rapidly reverts to its pre-compression state once compressive forces are removed from the device. Once the structural support layer is set in a particular location within the device it will return to that location after compressible forces are removed from the device and importantly will assist in forcing and/or enabling other layers within the device that are made of much less resilient material to be re-arranged to their pre-compressed state once compressive forces are removed from the device. In a preferred arrangement the structural support layer is resiliently biased against a rigid component of the device e.g. tray or holders and/or may be fixed to these rigid components; this aids in retaining the structural support layer at the desired location within the device and also assists in its function. The structural support layer may be made of any material that may be resiliently deformed and engineered to comprise a plurality of large holes without significantly impacting its resilient properties. Suitable materials for use as the structural support layer may comprise engineering plastics, polymers and composites and other similar materials and mixtures thereof that are able to impart the required resilient deformable properties to the support. Examples of suitable materials include: Polyethylene (High Density—HDPE); Polyethylene (Low Density and Linear Low Density—LDPE and LLDPE); Polypropylene PP; Polyamide (Nylon) PA (PA 6 and PA 12); Polytetrafluoroethylene PTFE; Polycarbonate: PC; Acrylonitrile Butadiene Styrene: ABS; Polystyrene (General Purpose—GPPS); Polystyrene (Hi Impact—HIPS); Polyethylene Terepthhalate (Polyester-PET); Polybutylene Terephthalate (Polyester—PBT); Thermoplastic Elastomer: TPE; Polyvinyl Chloride: PVC; Styrene Acrylonitrile: SAN; Polyphenylene Ether: PPE; Polyphenylene Sulphide: PPS; Polyphenylene Oxide: PPO; and Acrylonitrile Styrene Acrylate: ASA, or mixtures of one or more of these materials. All these plastics can be blended as well to get different properties, examples of suitable blends would be PP/PC, PA/ABS, PPO/PP. All of these plastics and other suitable materials may also be re-enforced with carbon or glass fibers, typically with between 5 and 40 wt. % fibre. The material selected for the structural support layer must be resistant to and compatible with the liquid and/or gel materials used in the device. In a preferred, embodiment the structural support layer is located within the device between a) the reservoir and/or VLAP material and b) the contact liquid delivery layer within the device. In a more preferred embodiment the structural support layer is located within the device between a) the reservoir and/or VLAP material and b) the mediating layer and/or wicking layer of the device.

In all aspects of the present invention, especially the second aspect, porous reservoir materials as herein defined below may be used.

Preferred reservoir materials have a compression modulus within the range of 150 to 650 N·m⁻²·Δmm⁻¹ and most preferably from 250-350 N·m⁻²·Δmm⁻¹. Compression modulus in the context of this aspect of the present invention relates to the resistance to permanent or semi-permanent reduction in material thickness with applied pressure.

Compression modulus is determined by taking a sample of the material of known thickness (typically within the range of 5 to 10 mm) and applying known weights (typically between 20 to 200 g) to the surface of the porous material e.g. foam, which is compressed when the weight is deposited on the material surface. The weight is applied over an area of 5.03×10⁻³ m² for a period of 10 seconds after which the weight is removed and the material thickness is determined using a Shirley thickness gauge and compared to the thickness of the material prior to application of the weight.

The porous reservoir material should have a compression modulus that is not so high as to effectively prevent compression of the porous reservoir material during use in a delivery device with the resultant controlled displacement of compositions, such as cleaning fluids or gels, from the porous reservoir material e.g. foam. In addition, the porous reservoir material should not have a compression modulus that is too low. A low compression modulus would result in excessive displacement of compositions from the porous reservoir material as it would be susceptible to a large decrease in material thickness under relatively low forces and especially under the typical forces observed during use.

The porous reservoir material may be any porous material with the requisite properties and should have an interconnected pore network wherein individual pores are connected to others such that liquids can easily flow through the entire structure, displacing air that may be present. It may be a woven or nonwoven porous material or a foam or any combination of two or more of these materials. The porous reservoir may comprise a composite material and/or may comprise a multilayer material. Preferably the porous reservoir material comprises one or more layers of porous material and preferably comprises one or more layers of foam material, preferably an open cell foam, e.g. reticulated foam. Preferably the porous reservoir material comprises a hydrophilic polyurethane open cell foam. Preferably the porous reservoir material comprises two or more layers of porous material and preferably comprises two or more layers of a hydrophilic polyurethane thermoset foam. Preferably the two or more layers of porous material are of the same thickness. One example of a suitable foam is Type 562-B as manufactured and supplied by Rynel Ltd. Co., Boothbay, U.S.A. Other suitable and preferred foams are those as described and prepared in WO 2005061600, the complete disclosure of which is hereby incorporated by reference. Thus the foam may be a low-density, open-cell, thermoplastic, absorbent foam, comprising at least two of the groups consisting of: a base resin, a surfactant, a thermoplastic elastomer, and a plasticizing agent and may be made by a method comprising the steps of: providing a foam polymer formula including the base resin, the plasticizing agent, and the surfactant; heating the foam polymer formula to create a polymer melt utilizing a blowing agent; foaming the polymer melt to a density of about 0.1 g cm⁻³ or less; and extruding the polymer melt to form an open-cell, soft, flexible, thermoplastic, absorbent foam.

Preferably, the porous reservoir material has a void fraction of greater than 80%, preferably greater than 85% and most preferably greater than 90% as determined by gas pycnometry. Preferably the porous reservoir material has a density of 0.2 g cm⁻³ or less, more preferably 0.15 g cm⁻³ or less and most preferably 0.10 g cm⁻³ or less as determined by ASTM D1622-98. The thickness of the porous material comprising the porous reservoir may be greater than 10 mm. The total thickness of the porous material comprising the porous reservoir is preferably less than 12 mm, preferably less than 11 mm and most preferably less than 10.5 mm. Most preferably it is within the range of 7 to 10 mm with a Coefficient of Variation of less than 5%. Typically, and preferably the reservoir material will have pores that are equally aligned in all planes x, y and z.

Preferably the porous reservoir material has a relatively high resiliency in combination with a compression modulus within the range of 150 to 650 N·m⁻². 4 mm⁻¹ and most preferably from 250-350 N·m⁻². 4 mm⁻¹. Preferably the resiliency as determined by ASTM D3575 is greater than 85%, more preferably greater than 90%, more preferably greater than 95% and most preferably within the range of 99-100%. Preferably the porous reservoir material exhibits a percentage strain as measured at 6.8 kPa by the method described in ASTM D3575 of 30% or less preferably 28% or less and most preferably 27%. Preferably the porous reservoir material has a mean flow pore diameter within the range of 80 to 400 microns.

The porous reservoir may comprise a composite material and/or may comprise a multilayer material. A nonwoven material may be composed of a variety of materials, such as, cellulose pulp or other absorbent fibrous material, capable of holding liquid within and between the pores of adjacent fibres. The porous reservoir preferentially has high capillarity, as characterized by a wicking height of over 10 mm, most preferably over 50 mm (test medium: water) when the material has its largest dimension in the vertical orientation, as this is especially advantageous when the device is in use in a vertical position. In a further alterative the porous layer may comprise a plurality of chambers each containing a porous material as hereinbefore described.

In certain aspects of the present invention wicking layers may be used.

In devices of the present invention porous reservoir materials or the vertically lapped nonwoven materials preferentially have high capillarity as this is especially advantageous when the device is in use especially in a vertical position. The wicking layers typically have a capillarity that is greater than that of the porous reservoir or the vertically lapped nonwoven materials. The porous reservoir layer or vertically lapped nonwoven material may preferably be contiguous with the wicking layer, which preferably is adjacent to and may be contiguous with the liquid contact delivery layer.

When used the wicking layer in this device has two important functions. The first is to moderate or reduce the forced flow rate of liquid/gel from the reservoir to the contact liquid delivery layer and thereby control the volume of liquid delivered to that layer. The second is to act as a wicking layer, which equalises the concentration of liquid across the working area of the device such that liquid is uniformly distributed immediately adjacent the contact liquid delivery layer even when acted upon by gravity. The reservoir layer or vertically lapped nonwoven layer is compressible and may see as much as a 50% or higher reduction in volume when compressed, (i.e. 10 mm+can be reduced to <5 mm when compressed). Some of the liquid is forced out of the reservoir in to the wicking layer when the device is compressed. This is possible because the reservoir consists of interconnecting pores that communicate with the surface of the material of the reservoir layer. Some of the liquid expelled from the reservoir will be forced in to the wicking layer. However, because the wicking layer is thin compared to the reservoir and the rate of forced flow in to the wicking layer is high, the wicking layer reaches absorbent capacity quickly and any excess liquid will then pool in the bottom of the device. As the user removes the pressure from the device, this excess liquid is then reabsorbed back in to the reservoir and the wicking layer. Thus this function is to moderate the total volume of liquid that is transported to the contact layer when the delivery system is activated by the user. The wicking layer acts a flow resistor when forced flow of liquid is induced. When the delivery system is compressed by the user, liquid can be expelled from the reservoir and transported through the thickness of the wicking layer toward the contact layer. Drag forces created as the liquid passes around the multiple fibre surfaces in the wicking layer generate drag forces that resist the flow in this direction. The flow resistance induced by the wicking layer (through-thickness) ensures that excess fluid is not transported out of the delivery system on each activation. This ensures a more effective and efficient use of the liquid agents used with the device.

Preferably the wicking layer comprises porous material. Preferably the wicking layer comprises a woven or nonwoven fabric, most preferably a nonwoven fabric. The porous material of the wicking layer is preferably comprised of a pore structure that is capable of retaining and distributing in-plane (i.e. in the x and y planes), the cleaning fluid/gel by means of capillary forces. The predominant direction of fibre orientation in the porous wicking material is preferably aligned with the longitudinal axis of the delivery device, with preferably greater than 80%, more preferably greater than 90% of the pores aligned in the x-y plane. When the device is compressed, the porous material of the wicking layer is such that it restricts or moderates the total volume of cleaning fluid/gel that may be evacuated from the reservoir and passed transversely through the wicking layer and to the exterior of the device via the liquid contact delivery layer. In this way, over-delivery of cleaning fluid/gel to the exterior surface of the contact liquid delivery layer is reduced or prevented.

When the wicking layer comprises a woven or nonwoven fabric the transverse flow resistance of the wicking layer is partly influenced by the total surface area of the constituent fibres, which may be adjusted by selecting fibres having different diameters and/or cross-sectional shapes.

It is preferred that the wicking layer comprises fibre surfaces that are wetable, by the composition to be discharged by the device e.g. cleaning fluids or gels and the like. In a preferred embodiment when the composition is aqueous the fibre surfaces are hydrophilic. Preferably the wicking layer has a sessile drop angle of less than 90° and more preferably less than 30°.

Suitable wicking materials include those composed of regenerated cellulose (specifically viscose, wood pulp, lyocell or Tencel®) which may be blended with synthetic materials (specifically PET, PA, PLA, PP or PE), wherein the synthetic component represents less than 40% by weight of the entire wicking material; plasma-treated aromatic and aliphatic polyesters and polyolefins; and blends thereof.

It is preferred that the wicking layer is a nonwoven fabric comprising a proportion of hydrophilic fibres or hydrophobic fibres that are surface modified using any method known in the art (e.g. plasma treatment, fibre finish, masterbatch additives) to enable them to be wetted by water and alcohol formulations (the latter including ethyl alcohol, i.e. ethanol). Inherently hydrophilic fibres in the art are composed of natural materials such as cellulose in native fibre form, e.g. cotton, flax, hemp, ramie, etc. or cellulose in regenerated form, e.g. lyocell (Tencel), viscose rayon, etc.

Preferably the wicking layer is a nonwoven fabric formed using a drylaid web formation method, such as carding wherein the fibre orientation can be controlled during production to enable the directional permeability and capillarity of the structure to be adjusted if required. In a particularly preferred embodiment, the fabric is produced from parallel-laid carded webs or cross-lapped carded webs wherein there is preferential fibre orientation in the machine direction (parallel-laid carded webs) or the cross-direction (cross-lapped webs) after bonding. Bonding is accomplished by a mechanical bonding technique. Suitable mechanical bonding techniques include hydroentangling (spunlace) and needlepunching or combinations thereof. Furthermore, the resulting fabrics preferably exhibit anisotropy in both permeability and capillarity as a result of preferential fibre orientation in one or more directions. Most preferably this is in the x-y plane of the wicking layer. When assembled in to the delivery device, the direction of predominant fibre orientation in the fabric is aligned with the long axis of the device, or depending on orientation of the device in a suitable direction to oppose gravity.

An embossed pattern may be applied to the fabric as part of the hydroentangling process producing areas of variable density. This is achieved by means known in the art using structuring support surfaces in the machine. A honeycomb pattern is particularly preferable in assisting with liquid distribution, since the differences in density also result in differences in permeability and capillary pressure.

A particularly suitable and preferred material is a nonwoven fabric comprised of 100% Tencel® fibre prepared by carding, cross-lapping followed by needle punching. Preferably, this fabric has a mean thickness of between 0.5 and 2.0 mm, more preferably 1.0 to 2.0 mm, preferably, 100% Tencel 1.7 dtex (linear density), 38 mm (mean fibre length), basis weight of the fabric: 80-100 g/m².

Another preferred material is a 65% Viscose rayon/35% Polyester, 100 g/m², having thickness range: 1-2 mm. The fabric is produced by carding wherein the webs from two or more carding machines are deposited one on top of the other (without cross-lapping) and then mechanically bonded by hydroentangling. Preferably the nonwoven fabric has a bubble point pore diameter of between 50 to 100 μm, more preferably 60 to 90 μm, more preferably 70 to 80 μm and most preferably about 75 μm. A Preferably the nonwoven fabric has a mean flow pore diameter of between 15 and 30 μm, more preferably 20 to 25 μm and most preferably about 23 μm. It is preferred that the wicking layer is from 0.5 to 3.0 mm in thickness, more preferably 0.5 to 2.5 mm in thickness and most preferably 1 to 2.0 mm in thickness. Preferably the wicking layer material has a mean flow pore diameter of between 10 to 100 microns.

The key distinctions between the mediating layer and the wicking layer as described is that the mediating layer is non-absorbent, whereas at the wicking layer is absorbent and the mediating layer is of significantly lower density, at 60 g·m⁻² or less, than the wicking layer which is from 80-100 g/m².

The contact liquid delivery layer may be a liquid permeable or impermeable film or membrane. Preferably the contact layer is made of liquid impermeable material. The film may be a porous film or a perforated film, e.g. a micro perforated film. The contact liquid delivery layer is preferably, a microperforated film. A variety of films may be used, thus, the film may be selected from any conventionally known film-forming polymers or blends thereof. Preferably, the film will be elastomeric and therefore have elastic properties, i.e. preferably a high elastic recovery (>75% at 1% extension). Preferably the film will be compatible with well-known printing techniques. Also, the film will be non-degradable and/or soluble in water or when in contact with an alcohol or an oil-water emulsion. It is especially importantly that the printed surface should be insensitive to alcohol, e.g. ethanol, which forms the basis of many commercially available antibacterial formulations. The contact liquid delivery layer preferably comprises valve like pore or slits.

The perforations in the liquid contact layer as slits and/or pore openings may vary depending, inter alia, upon the nature and composition of the antibacterial formulation present in the porous reservoir layer. Thus the slits and/or pores of the microperforated film may consist of sub-micron dimensions, however, preferentially, a microperforated film with openings of from 20-500 μm diameter (as measured across their minor axis) may be used, that is for example more than 50% of the pores have a diameter in the range of from 20-500 μm, preferably more than 70%, more preferably more than 90%. It is particularly advantageous if the liquid contact delivery layer, e.g. the perforated film is elastic so that the slit and/or pore openings may further open to their maximum extent due to the forces introduced by the user on this surface during use and the displacement of the liquid/gel from the porous reservoir layer and then self-close by elastic recovery of the film when the forces are removed from the system and the liquid flow ceases. Nevertheless, it should be understood that perforated high modulus films may also be suitable. Furthermore, it may be advantageous for each of the layers in the multi-layer device to possess elastic properties.

The elastomeric nature of this layer in combination with the use of closed slits and/or pores and material selection for the layer has distinct benefits for the devices of the present invention. The elastic contact liquid delivery layer of the present invention has two important functions. These combine to provide a layer with valve like pores or slits.

Firstly, the elastic contact liquid delivery layer is generally a dry-state, non-porous and non-permeable elastomeric film material. In place of the pores of the prior art, which are open structures, the film of the present invention preferably comprises closed slits and/or pores. The elastic contact liquid delivery layer with these properties acts as the final barrier between the reservoir in the interior of the device, which contains cleaning fluids or gels that incorporate volatile fluids (e.g. alcohol) and the exterior of the device. The contact layer controls the evaporation of cleaning fluids or gels that incorporate volatile fluids. Typical prior art devices with open porous layers lose significant quantities of volatile fluids from their reservoirs via evaporation and have seeping or wet-state contact layers, which are not desirable. The elastic contact liquid delivery layer of the present invention prevents this evaporation and fluid loss through use of a non-porous non-permeable film with closed slits and/or pores and thus ensures that there is no significant seeping from the device and the contact surface remains dry.

Secondly, the elastic liquid contact delivery layer, as the final barrier between the reservoir and the exterior of the device, must also be able to allow the cleaning fluids or gels that incorporate volatile fluids to pass through it and to the exterior surface of the layer. This is typically achieved in the prior art by the introduction of open pores or relatively open pores that enable quantities of cleaning fluids or gels that incorporate volatile fluids to pass through the layer but have the disadvantage of providing an evaporation pathway through the layer. The elastic contact liquid delivery layer of the present invention having a plurality of closed slits and/or pores overcomes this problem. A slit in this context is defined as a rectangular rather than substantially cylindrical opening such that the edges are capable of communicating when the material is in an unstrained state. When in position for use but without any applied pressure to the surface of the device the slits and/or pores of the contact layer are closed. In the closed non-impact state, and due to their method of manufacture, the layer material at the slit site is distorted effectively forming an overlapping and puckered arrangement closing the slit. In this position evaporation through the layer is significantly reduced compared to that observed with conventional porous layers because the two edges of the slit remain in communication closing the slit. With slits the separation of the adjacent parallel edges of these slits to open the closed slit is induced by mechanical force or shear induced in the elastic contact liquid delivery layer during use when the layer is contacted or impacted by a human hand. Thus at the point of need and only at that point the slits are open and allow the passage of cleaning fluids or gels from the reservoir to and through the layer and to the contacted surface of the layer. This slit opening mechanism is a localized mechanism, meaning that areas in the contact layer that are remote from the point of contact are relatively unaffected when other slits are being opened during use. This means that slits remote from the impact site on the layer remain closed or relatively closed compared to those at the site of impact. This mechanism ensures that the maximum flux of cleaning fluids or gel through the layer is at the point of contact. When the applied force of contact or impact is removed from the contact liquid delivery layer the opened slits return to their original closed position and function; the elastic recovery of the layer re-introduces the material distortion and puckering at the slit site closing the slit. With closed pores a similar mechanism occurs. In the closed non-impact state, and due to its method of manufacture, the layer material at the pore site is distorted effectively forming an overlapping and puckered arrangement closing the pore. On impact in use this region of the layer is elastically deformed and the distortion and puckering is temporarily removed to open the pore. When the contact pressure is removed the elastic recovery of the layer re-introduces the material distortion and puckering at the pore site closing the pore.

The elastic contact liquid delivery layer is fixed at its outer extremities to a rigid member so that when force is applied substantially perpendicular to the surface of the unfixed areas, the layer is partially elongated to accommodate the perpendicular displacement. This elongation of the film causes a temporary separation of the adjacent parallel edges of any slits and/or pores that are in the vicinity of the applied force. The adjacent edges return to their original position after the force is removed in order that the film remains substantially impermeable to liquid or gas in ambient conditions.

The slits and/or pores may be arranged in any fashion within the liquid contact delivery layer. They may be arranged in a parallel fashion either from top to bottom of the device or from side to side of the device within the liquid contact delivery layer. The slits may be arranged in a twill pattern. The slits may take the form of an x/y or other form of crossed slit.

Preferably to ensure that the parallel edges of the slits remain in the closest proximity, no stored strain is present in any direction within the layer after its integration into the device; this is especially beneficial when the slits are arranged in a twill fashion

Preferably, there is no pre-strain transverse to the slot length after its integration into the device. In some embodiments an amount of pre-strain of preferably 0.2%-5% of the relevant dimension in the layer in a direction parallel to the slot length may be advantageous in ensuring tight closure of the slots through additional contact pressure induced at the parallel slot edges.

Any non-permeable non-porous elastic material may be used as the elastic contact liquid delivery layer material. Preferably the layer is manufactured from an elastomeric polymeric material. By non-permeable is meant non-permeable to the typical components of the compositions located within the reservoir and used in the manufacture of cleaning fluids or gels that incorporate volatile fluids. The material may be a hydrophobic polymer, such as polyurethane (PU), polyethylene (PE), polypropylene (PP) polyamide (PA) or polyethylene terephthalate (PET) and copolymers thereof. The contact liquid delivery layer may comprise of a bi-layer in which the upper and lower layers are formed from different polymers. Further examples of suitable materials for use as the contact liquid delivery layer include but are not limited to one or more of polysiloxane, vinyl methyl silicone, chlorosulphonated polyethylene, TPE (thermoplastic elastomer), TPU (thermoplastic polyurethane), TPV (thermoplastic vulcanizates), TPO (thermoplastic polyolefin), TPE-E (thermoplastic polyester elastomers), TPC-ET (thermoplastic copolyester elastomers), TPE-A/TPA (thermoplastic polyamide elastomer), SBC (styrenic block co-polymers), SBR (Styrene butadiene rubber), Silicone, Polyisoprene, HNBR (Hydrogenated Nitrile Butadiene Rubber), EPDM (ethylene propylene diene monomer (M-class) rubber), NBR (Acrylonitrile Butadiene Rubber) XNBR (Carboxylated Nitrile Butadiene Rubber), Polybutadiene or copolymers thereof.

One preferred class of elastomeric materials for use as or in the contact liquid delivery layer of the present invention are manufactured using blown film extrusion processes and are therefore blown film materials. Another preferred class of elastomeric materials are cast extrusion films.

One preferred class of elastomeric material for use as or in the contact liquid delivery layer are multi-layered elastomeric materials with two or more layers, preferably three or more layers. In these multilayered materials the main or core layer may be any suitable thermoplastic material as herein described and the additional layers may also be elastomeric thermoplastic materials or may be polymeric layers of other types and composition. It is preferred that these multi layered elastomeric materials are manufactured using blown film extrusion processes or may be manufactured using a combination of blown film extrusion processes and other techniques such as coating and/or lamination.

In a preferred embodiment the contact liquid delivery layer comprises at least three layers of material. The core layer comprising one or more polymeric materials, preferably one or more thermoplastic elastomeric materials and the top and bottom layers comprising one or more polymeric materials and wherein the top and bottom layers are of identical composition but of different composition to the core material. It is envisaged that there may be some common materials to both the core and the top and bottom layers but that the overall composition of the core layer is different from the other two identical layers. Preferably this structure is manufactured using blown film extrusion techniques. In a further embodiment this blown film material may then be provided with one or more additional layers upon one or more of its two surfaces using additional techniques such as extrusion, casting, coating and/or laminating of polymer materials onto one or more surfaces. This or these additional layers may be deposited on the surface of the blown film material in order to improve one or more properties required for the devices of the present invention. The additional material may provide improved bonding properties to the backing layer or tray unit during manufacture of the device whilst not compromising or significantly comprising the other desired properties of the contact liquid delivery layer. In a further scenario the added material may be advantageous in improving the printability of the exterior surface of the device again whilst not compromising or significantly compromising the other film properties.

Suitable multilayered contact liquid delivery layer material may comprise one or more thermoplastic elastomeric materials as herein previously described as the core and top and bottom layers comprising one or more polyolefin materials with optionally one or more other polymeric materials such as polyethylene terephthalate (PET). The polyolefin materials may be preferably one or more of polyethylene (PE), polypropylene (PP), low density polyethylene (LDPE), linear low density polyethylene (LLDPE) and olefin co-polymers. The preferred elastomeric materials for the core comprise one or more of polyurethane (PU) based elastomers, elastomeric styrenic block copolymers (SBC), such as for example styrene-butadiene-styrene (SBS) and styrene-isoprene-styrene (SIS) block co-polymers, thermoplastic polyester elastomers or ethylene vinyl acetate (EVA) elastomers, The multilayered contact liquid delivery layer is preferably of overall thickness (as determined according to DIN 53 370) of from 50 to 300 microns, more preferably from 75 to 250 microns, more preferably 100 to 250 microns and most preferably 100 to 225 microns; with an ideal thickness of about 200 microns. Within this overall thickness each of the individual layers may be from 1 to 290 microns (for overall thickness of 300 microns), preferably for an approximately 100 micron multilayered material the top and bottom layers may be from 1 to 10 micron, preferably from 3 to 5 micron, with a core layer of from 80 to 98 micron with each thickness selected to provide and overall combined thickness of about 100 microns. For an approximately 200 micron multilayered material the top and bottom layers may be from 1 to 45 micron, preferably from 3 to 40 micron, more preferably 3 to 20 micron and most preferably 3 to 5 micron with a core layer of from 110 to 198 micron with each thickness selected to provide and overall combined thickness of about 200 microns. The preferred 100 micron film preferably has top and bottom layers of about 3 to 5 micron and a core of about 90 to 96 micron. One preferred 200 micron film preferably has top and bottom layers of 40 microns and a core of 120 microns. In a further preferred embodiment the core is of about 190 to 194 micron with top and bottom layers of 3 to 5 micron of PP/LDPE. In a preferred embodiment the elastomeric core material is a co-extruded mixture of SBC and EVA elastomers and the top and bottom layers are co-extruded polyolefin mixtures of LDPE/PP, with the while film preferably manufactured as a co-extruded blown film. A preferred monofilm material is a mixture of SBC and EVA elastomers. The monofilm or the multilayered film may be subsequently coated on one or both surfaces with polyolefin materials and preferably are coated on one surface with LDPE. In respect of the monolayer this may be one homogeneous polymer or polymer blend pf material. This monolayer may be manufactured from multiple layers combined to produce one integrated homogeneous material layer.

Preferably, the elastomeric film material has a maximum extension that is below the elastic limit of the layer material in order that on release of the impact force, up to 100% elastic recovery takes place so that the original positions of the slit edges and form of the slit are regained. This facilitates repeated, cyclic separation and recovery of the original slit edge positions and form. Closed pores are also able to return to their original pre-impact state.

The elastomeric film preferably exhibits a maximum extension of 500%-850% and a peak load at 20% extension of between 5 to 15N/25 mm and more preferably 7.5 to 12.5N/25 mm and most preferably about 10N/25 mm.

The breaking load of the film is preferably not less than 20 N/25 mm, more preferably not less than 30N/25 mm and most preferably not less than 40N/25 mm (8 MPa).

The elastomeric film is preferably less than 1000 microns in thickness, more preferably less than 800 microns in thickness, more preferably 50 to 1000 micron in thickness, more preferably 50 to 800 microns in thickness, more preferably 50 to 500 microns, more preferably 100 to 200 microns. It is preferred that the thickness of the elastomeric film or whatever structure is greater than 100 microns, preferably greater than 150 microns and most preferably greater than 175 microns to provide the maximum valve like properties for the film to ensure low evaporation losses during use.

The number of slits per square area in the elastic layer is preferably between 40 to 150 slits/in² (6.2 to 23.3 slits/cm²), more preferably 50 to 140 slits/in^{t} (7.8 to 21.8 slits/cm²), more preferably 60 to 130 slits/in² (9.3 to 20.2 slits/cm²), and most preferably 63 slits/in² (9.8 slits/cm²)-126 slits/in² (19.5/cm²). It is preferred that the slit length is 5 mm or less, more preferably 4 mm or less, more preferably 3 mm or less and most preferably 1.5 mm or less. Preferably the slits are within the range of 0.1 mm to 1 mm in length. The width of the slits is ideally as small as possible so that opposing sides of the slits are in contact when the elastic layer is not under any applied stress during use. There may be a small amount of separation and this may result in a slit width, which is preferably within the range of 5 to 200 μm, preferably 5 to 150 μm, more preferably 10 to 150 μm, more preferably 15 to 150 μm, more preferably 20 to 150 μm and more preferably less than 100 μm, more preferably less than 50 μm and most preferably less than 20 μm. Closed slits are preferred to closed pores but the elastic contact liquid delivery layer of the present invention may comprise slits and/or pores. When closed pores are present they may be present in the same pore density as described for slits.

The slits may be imparted to an elastomeric film through use of a cutting tool that that has a plurality of blades that may be impressed into and cut through the elastomeric film, such as a stamp press. This may be undertaken in a continuous fashion by feeding a web or elastomeric film into an engraved or toothed slitting roller. Pores when introduced may be introduced by a suitable pin arrangement on for example a stamp press that punctures the film. The elastomeric film is designed to limit the evaporation of a 62% ethanol in water composition to a maximum of 5% after five days' exposure in an environment of 21° C. and 65% RH.

In respect of all aspects of the present invention the elastic contact liquid delivery layer is preferably printed or otherwise coloured. It is preferable that such printed or colouring of the surface is fast (resistant) to the cleaning fluid/gel components contained within the assembly, including alcohols. Emblems, motifs, brands, health messages and advertising information can be applied to the surface of the elastic contact liquid delivery layer by printing.

It has been found that the printing of the elastomeric material is particularly challenging. In a further aspect of the present invention to facilitate printing the elastic film can be subjected to pre-heating prior to the printing stage to drive out moisture. Preferably the pre-heating is at a temperature within the range of 150 to 180° C., more preferably 160 to 180° C. and most preferably at up to 170° C.±5° C. It is preferred that the pre-heating is for between 2 to 40 seconds, more preferably, 5 to 30 seconds, more preferably 10 to 25 seconds and most preferably at about 20 seconds 170° C.±5° C. The pre-heated film is cooled prior to printing. The pre-heating may be omitted if there is no moisture present that could inhibit the printing process. In a preferred process the elastomeric film layer is printed prior to slitting and/or perforating. As alternatives the surface of the contact liquid delivery layer to be printed is treated to improve its printability. This may be achieved by selecting the appropriate top and bottom layer material for use in the manufacture of a multilayered contact liquid delivery layer, by selecting a suitable material as an additional layer for depositing on the blown film material surface to be printed, by corona discharge treatment of the surface, by electron beam treatment of the surface during or prior to printing and/or by UV grafting techniques to modify the surface properties.

To ensure long-term stability and high clarity images, printing is most preferably achieved by ultraviolet (UV) printing, which relies upon the application of UV-curable inks to the surface of the polyurethane film.

The method for the manufacture of an elastomeric film with valve like pores and/or slits comprises applying a strain to an elastomeric material, while the elastomeric material is under strain inducing a plurality of pores and/or slits through the elastomeric material, and removing the applied strain to enable the elastomeric material to elastically recover closing the pores and/or slits. In a preferred embodiment the applied strain is below 20% strain, preferably below 15% strain, more preferably below 10% strain and most preferably between 1 and 10% strain. The process in using elastomeric material under strain ensures that the pores and/or slits have a certain dimension; when the strain is removed the elastomeric film with pores and/or slits relaxes under elastic recovery and the pores and/or slits and the material around them contract with this recovery to a smaller dimension and to a closed state with localized distortion of layer material around the pores and/or slits. In this closed state these pores and/or slits may act as valves; closed when the film is relaxed and opened when the film is under strain. The valve like pores and/or slits may have a specific form due to the method of manufacture and not seen with conventional slitting and/or puncturing. This takes the form of excess material around the slit or pore site that protrudes from the surface of the film in the relaxed state. The film essentially has two major surfaces. When the film is slit or punctured under extension the slitting or puncturing device contacts one of these major surfaces, pushes through the film material and on puncturing the film material is exposed at the other film surface. When the slitting or puncturing tool is removed and the film is relaxed excess film material associated with the slit and/or pore protrudes from the surface of the film that is remote from that contacted by the slitting and/or puncturing tool. It is this protruding excess material in combination with the films elastic properties that provides the valve like function to the slits and/or pores. The protruding excess slit and pore material may be located on the contact surface of the elastic contact layer or may be located on the surface that faces inwards into the device and towards the reservoir or wicking layer when present. It is preferred that the protruding excess slit and pore material is on the inwards facing surface so as not to interfere with the printing of the contact surface. In a preferred embodiment the layer comprises slits and not pores.

In all aspects of the present invention a backing layer or tray unit may be provided to the device that prevents penetration of active liquid there through and which prevents evaporation. This tray unit or backing layer is preferably a non-porous and non-permeable film composed of a hydrophobic polymer. Exemplary hydrophobic polymers include polyethylene (PE), polypropylene (PP) or polyethylene terephthalate (PET) and copolymers thereof. This tray unit may be molded, formed, cut, injection molded, vacuum formed, thermoformed, and die-cut or formed through other methods. The material used has good vapour barrier properties; suitable materials include PET, APET, RPET, PP, PE, HDPE, ABS and similar materials. This tray unit acts as suitable container for the liquid storage materials (VLAP and/or reservoir) for the mediation layer and/or wicking layer and the liquid or gel like materials. The tray unit is preferably capable of being thermally bonded to the contact liquid delivery layer.

The device of the invention may include means for attaching the device to a surface. This may comprise an attaching means arrangement or may comprise one or more adhesive surfaces. The delivery device may by design be arranged to attach to a surface or to attach to itself, by wrapping around a surface. In a preferred embodiment the attaching means comprises an adhesive, for example, the backing layer is coated or substantially coated with an adhesive layer, e.g. a pressure-sensitive adhesive, to enable fixation of the surface mountable delivery device to various surfaces. Such an adhesive layer may be applied to the backing layer or alternatively the backing layer may itself comprise an adhesive provided that such an adhesive is contiguous over the surface of the porous layer and prevents penetration of active liquid through and which prevents evaporation. The adhesive is preferably a pressure sensitive adhesive which may optionally be alcohol soluble, thus enabling it to be removed from surfaces when the delivery device of the invention is removed. The pressure sensitive adhesive may be an acrylic adhesive such as an acrylate ester copolymer adhesive formed by the copolymerization of 2-ethyl-hexyl acrylate, butyl acrylate and acrylic acid. Alternatively, the adhesive layer may be an adhesive such as polyvinyl alkyl ether adhesive.

The device may be attached to a surface via means of a holster, which is more rigid than the tray unit component when used and is designed to hold the tray unit in place at the desired location for use and to support the tray unit in order to prevent it from bowing when pressure is applied by users to the surface of the device during use.

Thus in a further aspect of the present invention there is provided a device for storing and discharging liquid or gel like materials, which device comprises a tray unit and a holster for holding and securing the tray unit during use.

In this aspect of the present invention the tray unit may be a tray base device as herein described and the holster is adapted to secure this device in the desired location and in addition is adapted as herein described to aid in the function of the tray unit. The holster may comprise features that co-operate with features on the device to ensure effective discharge of material from the device. Further description of the holster and its features and those of the tray unit are provided below.

In all aspects of the present invention it is preferred that the contact liquid delivery layer and the tray unit or backing layer may be capable of thermoplastic bonding, e.g. ultrasonic, radio frequency joining and heat welding.

In use, the devices of all aspects of the present invention may and preferably do contain one or more active agents. The vertically lapped nonwoven storage material, and when present the porous reservoir or the wicking layer is impregnated with any conventionally known active agents, such as, an active agent selected from one or more of an antimicrobial agent, a medicament, a cosmetic a perfume, and a deodorant. In a preferred embodiment of the present invention the active agent is an antimicrobial agent. The active agent may be present in a form selected from form selected from solid, liquid, gel, suspension or emulsion and may be microencapsulated. Preferably, the active agent will be present in liquid or gel form. The term antimicrobial will be well understood by the person skilled in the art and shall include antibacterial, antifungal and antiviral compositions; and mixtures thereof. The term antibacterial shall include bactericidal and bacteriostatic compositions.

Any conventionally known antimicrobial composition may be used, most preferably, disinfectants are used, for example, alcohols, such as “surgical alcohols”, e.g. ethanol, 1-propanol and 2-propanol/isopropanol; chlorhexidine (0.5-4% w/v) including alcoholic formulations, isopropyl alcohol (60-70% v/v), ethyl alcohol (80% v/v) with or without emollients, povidone-iodine (0.75-1%), peroxygen based on potassium peroxomonosulphate or mixtures thereof.

Other antimicrobial compositions which may be mentioned include, quaternary ammonium compounds, such as benzalkonium chloride, iodine, phenol (carbolic acid) compounds, peracetic acid or silver compounds; or mixtures thereof. A preferred antimicrobial composition is an alcohol, such as that commercially available as Cutan® from Deb Limited in the UK. An especially preferred antimicrobial agent has an alcohol content of from 58 to 78% w/w, preferably from 68 to 72% w/w and most preferably alcohol content of 70% w/w.

It is an especially preferred feature of all aspects of the present invention that the device is adapted to remain bacteriostatic, fungistatic and viristatic during its lifetime.

A further preferred antimicrobial agent is one which is capable as acting as bactericidal agent or bacteriostatic agent, e.g. to MRSA. The antibacterial agent is especially a bactericidal agent or a bacteriostatic agent to one or more of MRSA, MSSA, Necrotizing fasciitis, Escherichia coli, NorA, Clostridium difficile, Norovirus, Enterococcus faecium and pseudomonas aruginosa. For example, vancomycin, methicillin, etc.

Examples of antifungal agents include, boric acid, or combined antibacterial and antifungal agents, such as triclosan.

The total loading of antimicrobial agent in the porous reservoir layer is dependent upon, inter alia, the form, thickness and density of this layer. This determines the total pore volume or porosity of the layer and therefore its absorbent capacity. The delivery rate may be controlled by the compression resistance of the material used, the total loading of the active agent, e.g. liquid, in conjunction with the properties of the wicking layer when present and the contact liquid delivery layer and the viscosity of the liquid. The latter can be controlled by additives, such as a thickener, if required.

In all aspects of the present invention the contact liquid delivery layer with or without physical features that prevent full contact by the human or object with the contact surface may also be provided with a removable cover layer as protection. Similarly, if the backing layer is provided with an adhesive layer then the adhesive layer and/or the backing layer may also be provided with a cover layer. The removable cover may be, for example, a silicone coated release paper. Alternatively, the whole delivery device may be presented in a sealed package.

The delivery devices of all aspects of the present invention may be made up in a variety of forms. By way of illustration, such forms include, but shall not be limited to, stickers, tapes, pads, tubes, sleeves, socks, wipes, cleaning devices and the like. The device may be surface mountable on a vertical surface or any other orientation or surface and may be wrapped around handle form surfaces as open or closed tubes. Other applications include agricultural pads or mats such as for example foot and mouth infection pads or mats, passenger foot pads at airports, trolley pads on hospital doorways, handles for hospital doorways, beds or other hospital equipment and all points of patient or health professional or public contact such as for example hospital theatre mats, hospital beds, cupboards etc., lifts, handles on buses/trains and other forms of public transport, seats, food preparation surfaces, etc.

In a further aspect of the present invention we provide a method of preventing the transmission of microorganisms which method comprises the use or application of a delivery device as hereinbefore described in each aspect or combination of aspects of the present invention.

In a further aspect of the present invention we provide a method of discharging a liquid or gel like material which method comprises the use or application of a delivery device as hereinbefore described in each aspect or combination of aspects of the present invention.

During manufacture of the device it is preferred that the form of the vertically lapped nonwoven storage material selected is oversized compared to the tray unit compartment into which it will be placed. It is preferred that the vertically lapped nonwoven storage material is impregnated with liquid or gel like material and is then longitudinally compressed to into the smaller tray unit compartment of the device. This oversized form of vertically lapped nonwoven storage material and sequence for manufacture is preferred for two reasons. Firstly, in the non-compressed state the vertically lapped nonwoven storage material may be more easily impregnated with the liquid or gel like material and secondly when in a compressed state in the tray unit compartment the compressed vertically lapped nonwoven storage material is at the desired density for use and is less shrinking or settling of this material in the finished device and during use. Thus the present invention also provides for a device in accordance with the first aspect of the present invention where the vertically lapped nonwoven storage material is in a compressed state within the device. Preferably the density in the compressed state is greater than 40 kg·m⁻³, more preferably greater than 45 kg·m⁻³ and most preferably 50 kg·m⁻³.

The invention in all its aspects will now be referred to by the following Figures, which show/represent various forms/states and designs, the invention could take and in which:

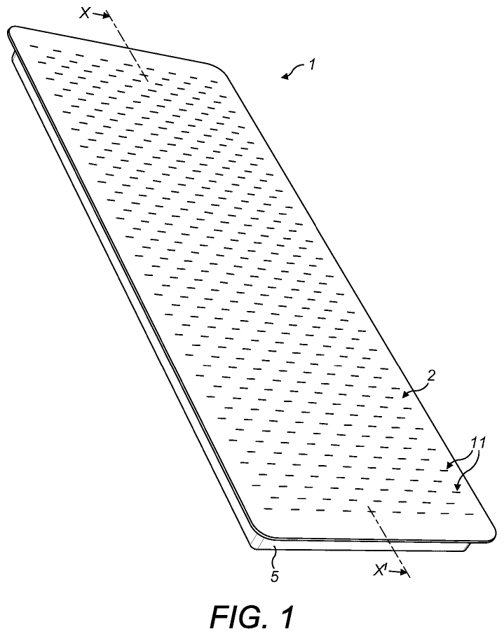

FIG. 1 is a perspective view of a delivery device according to a preferred embodiment of the present invention;

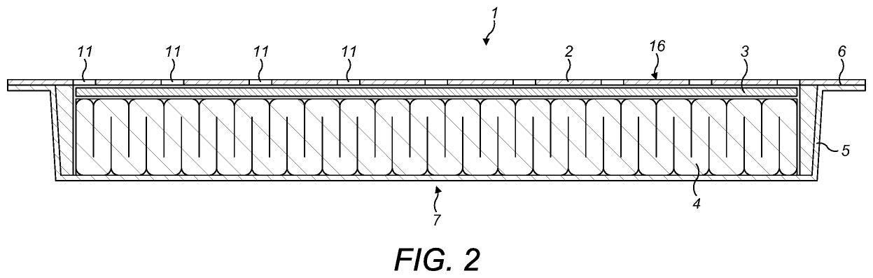

FIG. 2 is a schematic cross-section across plane x, x′ of FIG. 1 of a preferred delivery device of the present invention incorporating the first and second aspects of the present invention;

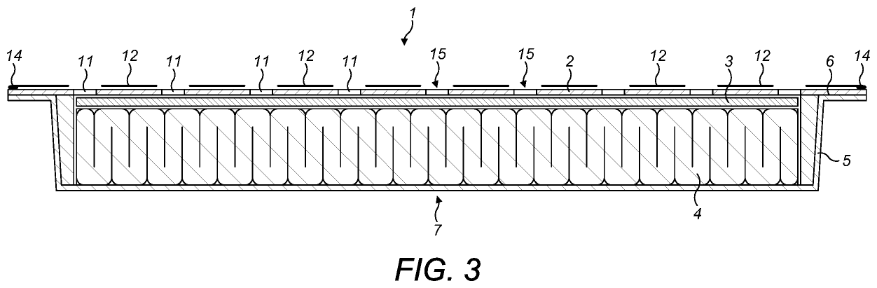

FIG. 3 is a schematic cross-section across plane x, x′ of FIG. 1 of a preferred delivery device of the present invention incorporating the first and second aspects of the present invention and illustrating the third aspect of the present invention;

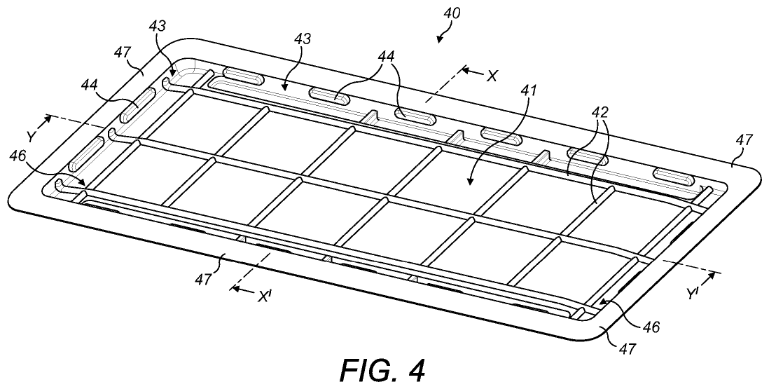

FIG. 4 is a perspective view of a tray unit for use in the device of the present invention;

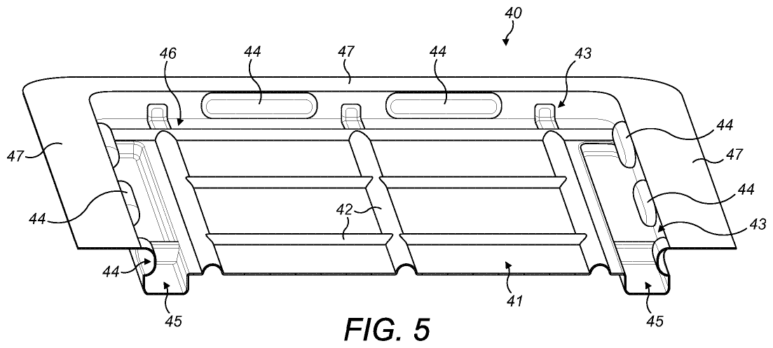

FIG. 5 is a cross-section across the plane x, x′ of the tray unit of FIG. 4;

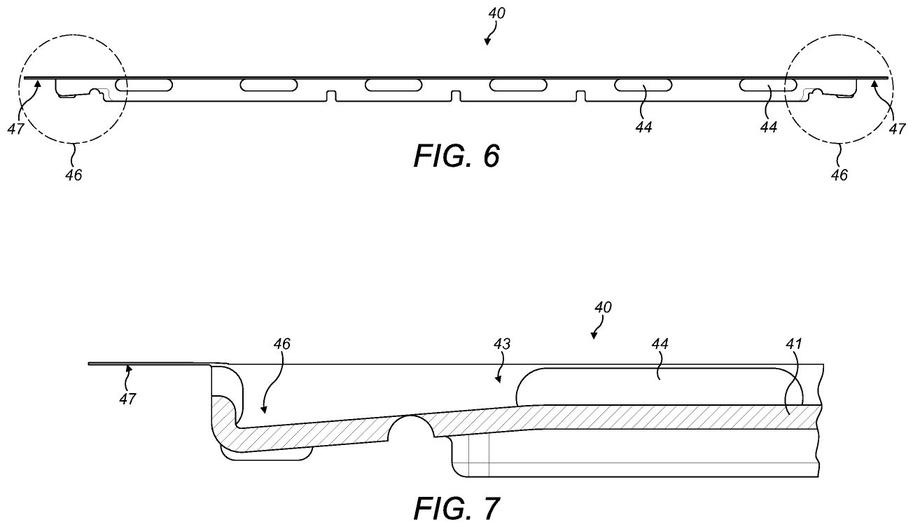

FIG. 6 is a side view of the tray unit of FIG. 4;

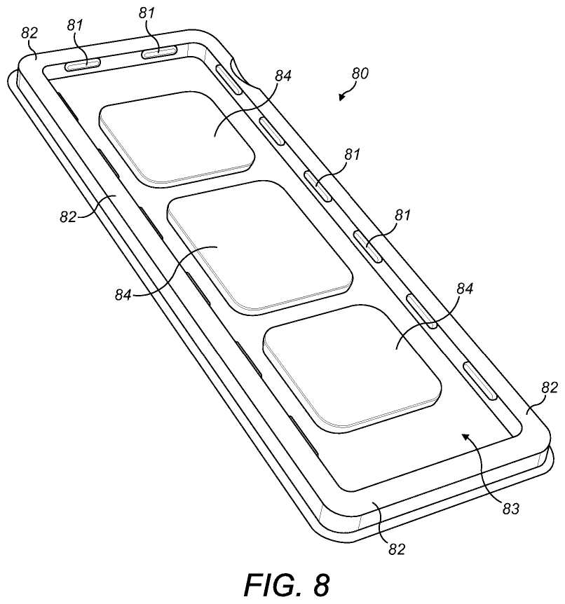

FIG. 7 is a cross-section across the plane Y, Y′ of the tray unit of FIG. 6;

FIG. 8 is a perspective view of a holster unit for use with the tray unit device of the present invention;

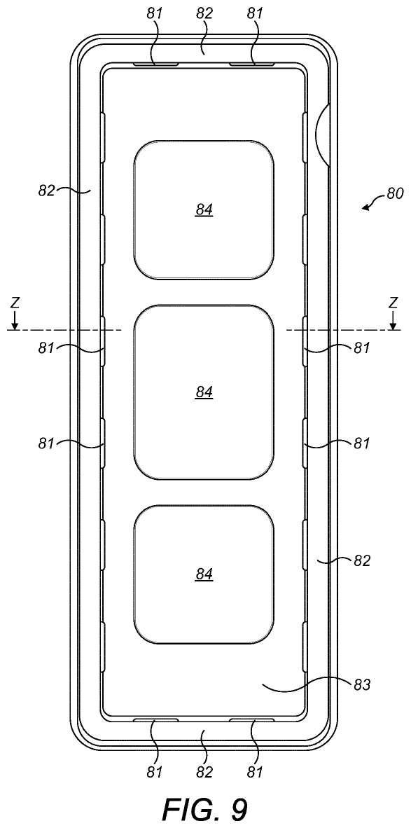

FIG. 9 is a plan view of a holster unit for use with the tray unit device of the present invention;

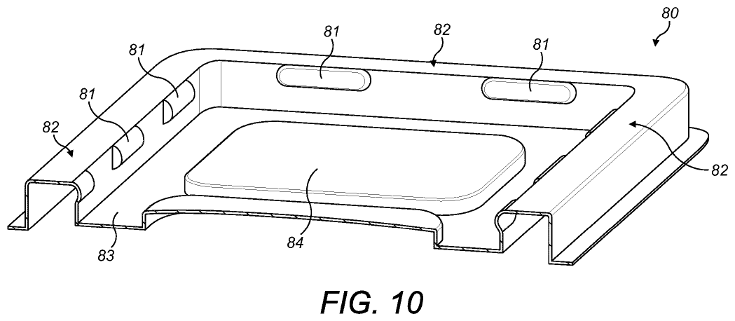

FIG. 10 is a sectional view along the axis z, z′ of the holster unit of FIG. 9;

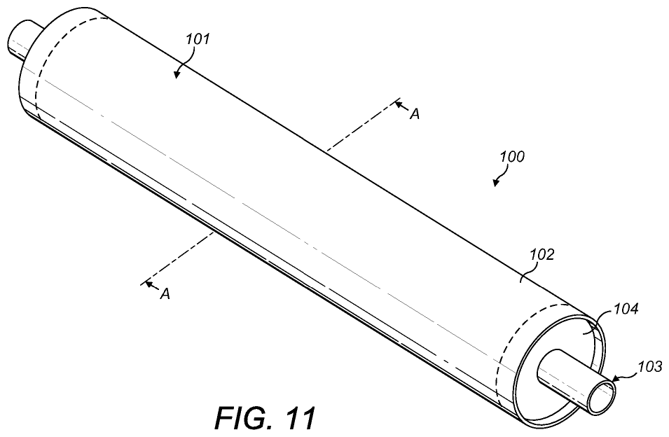

FIG. 11 is a perspective view of a tubiform device of the present invention suitable for use on a handle;

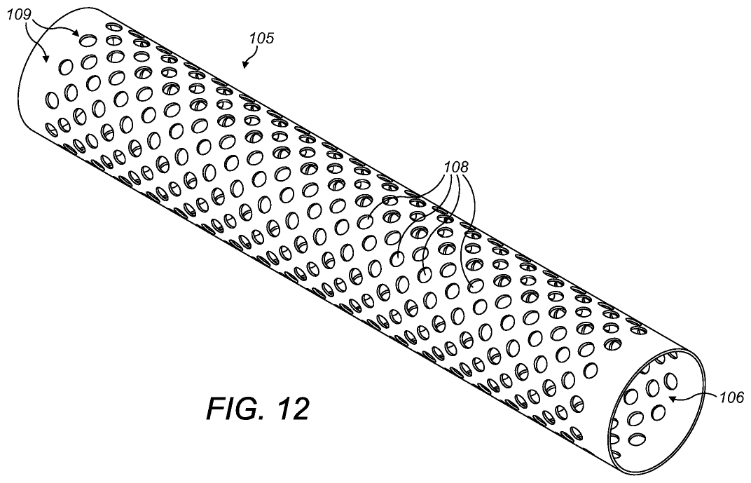

FIG. 12 is a perspective view of a structural support layer for use in a tubiform device; and

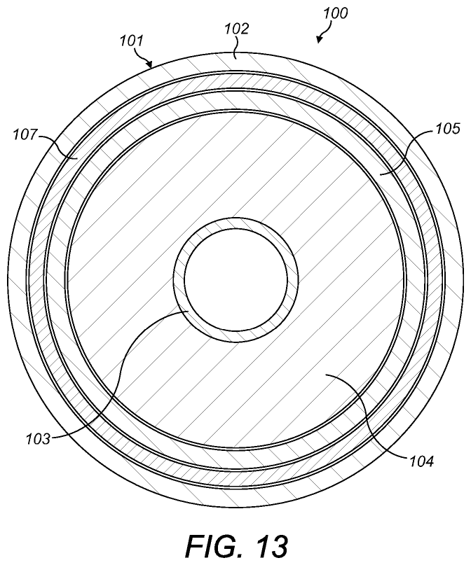

FIG. 13 is a schematic sectional view along axis A of the tubiform device of FIG. 11.

With reference to FIGS. 1 and 2 a deliver device (1) comprises a contact liquid delivery layer (2) adjacent to a liquid moderating layer (3), which is adjacent to a vertically lapped nonwoven material as the storage medium for the liquid or gel like materials within the device (4). The liquid moderating layer (3) and the vertically lapped nonwoven material (4) are located within a tray unit (5), which is bonded to the contact liquid delivery layer (2) at the periphery (6) of the tray unit (5) and the contact liquid delivery layer (2). The liquid moderating layer (3) is empty and the vertically lapped nonwoven material (4) is impregnated with an antimicrobial composition. The device may be attached to a vertical surface (not shown) by applying adhesive to the rear surface (7) of the device.

With reference to FIG. 3, there is shown the device of FIG. 2 with the addition of a physical feature (12) located upon the contact liquid delivery layer (2) and secured to the edges of the tray unit (5) at points (14). The physical feature (12) is shown in highly schematic form and in reality is in the form of a filamentous material that covers the contact liquid delivery layer (2). In this schematic form the physical feature (12) is illustrated with openings (15) over the slits (11) simply to illustrate that this physical feature (12) does not occlude the slit openings (11).

During use a person makes contact with the physical feature (12) and in doing so compresses the device (1). Before compression the slits (11) are closed and any antimicrobial composition is unable to exit through the surfaces (12) of the layer (2) which are impermeable. During compression the layer (2) is elastically deformed and under this deformation the slits (11) proximate to the deformation point and previously closed open and parallel sides separate to define a pore through which any antimicrobial composition may pass through the layer (2). It should be noted that slits that are remote from the point of compression are not opened by this mechanism as there are no local transverse forces across these slits to elastically deform the layer (2) proximate to these slits (11). When the compressive force is removed the layer (2) returns elastically to its original state and the parallel sides of the slits (11) close moving adjacent to each other and in doing so close the open pore. Under compression at contact the vertically lapped nonwoven material (4) is compressed and at the point of compression antimicrobial liquid passes from the vertically lapped nonwoven material (4) and through the liquid moderating layer (3), to pass through the elastically deformed slits at the point of compression and onto the contact surface (16) of the device (1). At the point of compression, the physical feature (12) prevents the compressing object e.g. human hand from occluding the slits (11) at the point of compression thus allowing the liquid to exit the device and onto the contact surface (16) and into contact with the compressing object. This arrangement ensures the optimum delivery of liquid material to the object contacting the device.

With reference to FIGS. 4 to 7 there is shown a preferred form of tray unit (40) for use in the device of the present invention. This tray unit is designed to hold and retain various components and ensure containment of the liquids or gels for dispensing when use in combination with the contact surface layer. In FIGS. 4 to 7 a tray unit (40) is shown which is rectangular in form and has a base (41) with longitudinal and lateral re-enforcing ribs (42) and four walls (43) generally upstanding from the base (41). At least two of the walls (43) comprise holster engaging means (44) and preferably as shown in FIG. 4 all of the walls (43) comprise holster engaging means. As illustrated in FIGS. 4 to 7 these engaging means make take the form of one or more slots (44) on the external surfaces of each wall, which generally protrude inwardly into the tray unit. When the tray unit (40) is engaged with the holster (80) these slots (44) engage with corresponding protrusions (81) in the holster (80). The tray unit (40) further comprises longitudinal channels (45) running along the longitudinal sides of the tray unit (40) and scalloped end regions (46), which will be described in more detail below. The longitudinal channels (45) provide additional rigidity. The top periphery of the walls (43) extend beyond the upstanding walls surface to form a peripheral flange (47), to which the contact surface layer of the device may be bonded during manufacture. This flange (47) is also of a form to preferably co-operate with tray flange contact surfaces (82) of the holster (80). As can be seen in FIGS. 5 to 7 the channel (45) and scalloped regions (46) have internal surfaces that are below the surface of the base (41). The scalloped regions (46) are therefore deeper than the majority of the base (41) area. When the vertical lapped nonwoven material is inserted into the tray unit (40) its top surface is at or below the flange (47) surfaces adjacent to these scalloped regions (46) compared to the flange (47) surfaces proximate to other regions of the base (41). This arrangement assists with assembly of the device by reducing the stretching of the contact surface layer at undesired angles over the vertical lapped nonwoven material of the tray unit (40) proximate to the flanges (47) at either end of the tray unit (40). They also assist in engaging the vertical lapped nonwoven material within the tray unit (40) during assembly of the device. The design of the tray unit (40) is such that the underneath surface of the base (41) is located above the bottom external surfaces of the longitudinal channels (45) and the scalloped regions (46).

With reference to FIGS. 8 to 10 there is shown a preferred form of holster unit (80) for use in the device of the present invention. The holster unit (80) is generally permanently or semi-permanently secured to a surface where the device of the present invention is to be located during use. This is for example a vertical surface on a door. The function of the holster unit (80) is to provide such a location for holding and securing tray unit devices (40) of the present invention that may easily be attached to the holster unit (80) at this location and easily removed and replaced when the need arises. The use of the holster unit (80) aids in the easy deployment and use of the tray unit devices (40) of the present invention. The holster unit (80) generally comprises a tray like structure into which the tray unit (40) may be securely and reversibly inserted. The exterior wall surfaces of the tray unit (40) contact the internal wall surfaces of the holster unit (80) and the slots (44) of the tray unit (40) are reversibly engaged with the protrusions (81) of the holster unit (80). This engagement is robust enough to hold the tray unit (40) firmly within the holster unit (80) during use but is such that when appropriate force is applied to the tray unit (40) it may be disengaged and removed from the holster unit (80). The base (83) of the holster unit (80) further comprises a series of raised plinths (84) that make contact with and support the base (41) of the tray unit (40) when it is in locked engagement with the holster unit (80). In addition, the flanges (47) of the tray unit (40) are supported by and in contact with the tray flange contact surfaces (82) of the holster unit (80).

With reference to FIG. 11 there is shown a tubiform device (100) of the present invention suitable for use on relation to handles e.g. door handles. The device (100) has an external contact surface (101) of the contact liquid delivery layer (102). Central to the device is an inner conduit member (103), which acts effectively both as a backing layer for the device and also provides the means by which the tubiform device (100) can be secured over a door handle or similar as a sleeve or engineered into other door handle arrangements. Also show in part is a key internal component of the device (100), namely the VLAP material in a tubiform arrangement (104). The tubiform arrangement of the device enables a human hand to grab the device and through compressive forces on the contact liquid delivery layer (102) actuate the device to expel liquid or gel from the VLAP storage (104), through the contact liquid delivery layer (102) and onto its external contact surface (101) having passed through the other layers present but not shown in this Figure; these additional layers will be discussed below.

With reference to FIG. 12, there is shown a structural support layer (105) as used in the tubiform device (100) of FIG. 11. This tubiform structural support layer (105) sits at a location in the device (100) that is between the contact liquid delivery layer (102) and the tubiform VLAP material (104). It is made of resilient but flexible material so that it may be deformed under compression and then revert to the form indicated when these compressive forces are removed. In reverting back to its original pre-compressed form it forces any layers external to it including the contact liquid delivery layer (102) back to their pre-compressed form. In addition, it also redefines the internal space within its bore (106), which accommodates the tubiform VLAP material (104) and in doing so enables the tubiform VLAP material (104) to decompress and accommodate this internal space (105) substantially in its original form. This arrangement therefore ensures effective and extended use of the device (100). The structural support layer (105) in addition to being resiliently deformable also has a plurality of large holes or passages (108) that communicate between its bore (106) and its external surface (109). These holes or passages (108) effectively allow substantially unrestricted expulsion of liquid or gel in the VLAP or reservoir material (104) located within the bore (106) from the VLAP or reservoir material (104) and to the external surface (109) and eventually out of the device (100) in a controlled fashion through other layers external to the structural support layer (105) and as described herein.

With reference to FIG. 13, there is shown a cross-section along axis A of the tubiform device (100) illustrated in FIG. 11. Indicated are: an external contact surface (101) of the contact liquid delivery layer (102); the central inner conduit member (103); the VLAP material in a tubiform arrangement (104); and the structural support layer (105) as described in relation to FIGS. 11 and 12. In addition is indicated a liquid mediating layer (107). In this arrangement the structural support layer (105) is located between the VLAP material (104) and the liquid mediating layer (107) and the contact liquid delivery layer (102).

### EXAMPLE

A device for storing and discharging liquid or gel like materials in accordance with the present invention was prepared as follows:

Vertically Lapped Nonwoven Material Storage Medium

A commercially available vertically lapped nonwoven material was used as the storage medium and manufactured and sold under the trade names VLAP and Struto. The material had a composition of 40% hydrophilic PET (4.4 dtex), 40% PET/CoPET BiCo (2.2 dtex) and 20% conjugate/spiral PET (10 dtex). This material had a density of 55 kg/m³, a thickness of 30 mm and was cut into 2 m length slabs. These slabs were converted into 3×10 mm thick slabs using band knife. These 9 mm slabs were then stamped into 270×71 mm pieces using bladed press, with longitudinal edge being in machine direction, perpendicular to the strata.

Mediating Layer

A hydrophilic polypropylene spunbond material of weight 20 gsm was selected as the mediating layer material.

Contact Liquid Delivery Layer

The material selected for this layer was a multilayered 200 micron material comprising a core of SBC/EVA elastomer film that had been coextruded with 5 micron polyolefinic printable surface layer. Individual reels of this material were processed through a perforating drum machine. The main perforating drum possess a 3D pattern of narrow blades of the dimensions and frequency of the desired perforation pattern. Inlet tension setting is 30/25 N, perforation pressure 650 psi and post perforation speed is +2%.

Tray Unit

A PET/PE film (400/50 micron) was vacuum formed over specifically designed 3D mould to produce multiple trays per cycle (depending on machine and mould size used). The tray unit was as described in FIGS. 4 to 7 and was designed to have a 3 mm internal depth to the tray base so that the top of the inserted vertically lapped nonwoven storage medium reservoir protruded above top surface of the tray walls; there was a 7 mm external depth to allow secure fastening into the holster unit. The tray unit also has internally scalloped ends to reduce the stretching of the perforated film/opening of the perforations when film is pulled at an acute angle over the top and bottom edges of the nonwoven reservoir in the tray. These internally scalloped ends also assist in the insertion of the gel filled nonwoven reservoir into the tray by preventing the short edges of the vertically lapped structure “springing” out of the tray, which would occur if the depth at each end was only 3 mm. The tray unit also has longitudinal channels, as well as longitudinal and lateral ribs, in the base to improve rigidity of the tray for processing, and in use.

Holster Unit

The holster unit was vacuum formed from 900 micron APET film. The internal cavity of the unit was 7 mm with fastening protrusions around the internal perimeter to secure tray unit once inserted. The holster unit also has 3×4 mm high rectangular raised plinths projecting upward into the cavity from the base to support the base of the shallower tray unit when inserted to prevent bowing of the tray unit when pressure is applied to tray unit in use.

Active Liquid Medium

The active medium comprised a viscosity modified 57% ethanol gel.

Device Assembly

160 g of active liquid medium gel was distributed uniformly throughout the stamped vertically lapped nonwoven pad (270×71 mm). This could be by impregnation through a fixed distance nip, multiple internal injectors, forced in via a scraper/doctor blade, or a combination of the above. The 270 mm long gel filled VLAP was compressed longitudinally into the 250 mm long internal tray unit cavity and the upper sealing surface of the tray (the upper surface of the flanges) was cleaned of fugitive gel to prevent this from interfering with heat sealing. An oversized VLAP slab is used for a number of reasons. Lower density material is easier to fill with gel and compressing gives the desired finished density. Also, compressing longer lengths of VLAP removes the possibility of the VLAP shrinking/settling longitudinally excessively in situ, therefore avoiding reservoir free cavities at the top or bottom of the finished pack. Consequently, the density of the VLAP once installed was 58 kg/m³. The tray filed with VLAP material was then inserted into a die to secure it during the heat sealing process.

A 250 mm strip of hydrophilic polypropylene spunbond internal localised mediating layer was placed flat, and in register with, the gel impregnated nonwoven VLPA layer within the tray unit. The perforated contact liquid delivery layer was held under tension (1.4 kg per 200 mm width), print over the tray located in the die with the gel impregnated VLAP in its internal cavity covered by the mediating layer. Heat sealer was then used to apply heat and pressure to the contact liquid delivery layer onto the tray sealing flanges (141° C. for 8 secs) to heat seal the underside of the elastic film to the upper surface of the tray unit flanges. The tray unit was allowed to cool before removing from the die to avoid distortion of the tray plastic while it is still hot and flexible.

The finished tray unit was then sandwiched between 2 layers of taut PET/Al foil/PE film (with PE facing in towards the unit). Heat and pressure are then applied just outside the perimeter of the unit to create a hermetic seal for the unit, therefore eliminating premature alcohol evaporation during storage.

For deployment the holster unit was securely attached by adhesive bonding to an upright contact surface of a door. The tray unit was removed from its packaging and placed into the holster unit with light pressure to lock the tray unit within the holster unit. During use the tray unit was found to deliver adequate and effective amounts of gel material to its contact surface during use and there was no significant pooling of gel material within the tray unit during its operation.

Throughout the description and claims of this specification, the words “comprise” and “contain” and variations of the words, for example “comprising” and “comprises”, means “including but not limited to”, and is not intended to (and does not) exclude other components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise. Features, integers, characteristics, compounds described in conjunction with a particular aspect, embodiment or example of the invention are to be understood to be applicable to any other aspect, embodiment or example described herein unless incompatible therewith.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

1-77. (canceled)

78. A device for storing liquid or gel materials and for discharging liquid or gel materials upon compression of the device; the device comprising vertically lapped nonwoven material as a storage medium for the liquid or gel materials within the device and a contact liquid delivery layer.

79. The device as claimed in claim 78, wherein the vertically lapped nonwoven material comprises fibers and a material base of the fiber comprises at least one synthetic polymer.

80. The device as claimed in claim 79, wherein the fiber comprises at least one synthetic thermoplastic polymer fiber.

81. The device as claimed in claim 79, wherein the material comprises a mixture of fiber types and/or fiber material compositions.

82. The device as claimed in claim 79, wherein the fiber component comprises one or more of polypropylene (PP), polyethylene (PE), polyamide (PA), PLA, PBT, PET, coPET, copolyester elastomers, HDPE, LDPE, PPS, PEI, PETG, PCT, elastomeric fibers or mixtures thereof, bicomponent fibers and/or conjugate fibers.

83. The device as claimed in claim 78, wherein the vertically lapped nonwoven has a density within the range of 20 to 90 kg·m⁻³ in the non-compressed state.

84. The device as claimed in in claim 78, wherein the vertically lapped nonwoven material is in a compressed state within the device.

85. The device as claimed in claim 78, wherein the vertically lapped nonwoven material has a weight within the range of 200 to 900 g·m⁻².

86. The device as claimed in claim 78, wherein a surface of the vertically lapped nonwoven storage material is closed and exhibits no open surface pore structure.

87. The device as claimed in claim 86, wherein a surface of the vertically lapped nonwoven storage material has a ratio of open surface area to total surface area no greater than 0.3.

88. The device as claimed in claim 78, wherein the vertically lapped nonwoven material is in communication with a liquid mediating layer located between the vertically lapped nonwoven material and the contact liquid delivery layer of the device.

89. A device as claimed in claim 78, further comprising at least one internal structural support layer.

90. The device as claimed in claim 78, wherein the contact liquid delivery layer comprises a multi-layered elastomeric material film with two or more layers.

91. The device as claimed in claim 90, wherein the multi-layered elastomeric material film comprises a top layer and/or a bottom layer comprising co-extruded polyolefin mixtures of LDPE/PP.

92. The device as claimed in claim 90, wherein the contact liquid delivery layer comprises SBC and EVA elastomers.

93. A device as claimed in claim 78, wherein one surface of the contact liquid delivery layer comprises a coating of LDPE.
